(19) United States
(12) Patent Application Publication
(10) Pub. No.: US 2012/0171169 A1
(43) Pub. Date: Jul. 5, 2012

(54) COMPOSITIONS AND METHODS FOR TREATING, PREVENTING, OR ALLEVIATING BONE OR CARTILAGE DISEASES

(76) Inventor: Jaewoo Pak, Seoul (KR)

(21) Appl. No.: 13/035,571

(22) Filed: Feb. 25, 2011

(30) Foreign Application Priority Data
Dec. 31, 2010 (KR) 10-2010-0140123

(51) Int. Cl. A61K 35/12; A61P 19/02; A61P 19/08

(52) U.S. Cl. 424/93.7

(57) ABSTRACT

The present invention provides pharmaceutical compositions, methods of preparations, and uses for treatments, preventions or alleviation of bone and cartilage diseases. The present invention discloses methods and pharmaceutical compositions comprising adipose tissue-derived stem cells, platelet rich plasma, calcium chloride, and hyaluronic acid for the treatments, preventions, or alleviation of bone diseases; methods and a pharmaceutical compositions further comprising of dexamethasone for treatments, preventions, or alleviation of cartilage diseases.

### TECHNICAL FIELD

The present invention relates to a pharmaceutical composition and a method for the treatment, prevention, or alleviation of bone diseases comprising adipose-derived stem cells, platelet rich plasma, calcium chloride, and hyaluronic acid for the treatment, prevention, or alleviation of bone diseases, and a pharmaceutical composition and a method further comprising dexamethasone for the treatment, prevention, or alleviation of cartilage diseases.

### BACKGROUND OF THE INVENTION

A degenerative arthritis, also known as osteoarthritis, is a disease causing pain due to wearing down of the articular cartilage, with aging and excessive usage. However, in recent years this disease is frequently being shown in young people. Conventionally, the damaged tissue or the whole damaged articulation is removed for the treatment of the diseases created by meniscus damage or degenerative arthritis. However, these types of surgery takes prolonged time for the recovery due to large surgical wounds.

Adipose tissue-Derived Stem Cells (ADSCs) have been widely used in Korea over the last few years by plastic surgeons as a semi-permanent volume expander. In June 2009, Korean Food and Drug Administration (KFDA) allowed ADSCs to be used as autologous cell transplant when obtained and processed within a same medical clinic with minimal processing.

Mesenchymal stem cells (MSCs) are found in numerous human tissues including bone marrow, synovial tissue and adipose tissue. These have been shown to differentiate into bones, cartilage, muscle and adipose tissue, representing a promising new therapy in regenerative medicine (Arnoczky S. P.: Building a meniscus. Biologic considerations. Clin Orthop Relat Res (367 Suppl): S244-53 (1999)). Because of their potent capabilities, MSCs have been used successfully in animal models to regenerate cartilage and bones (Barry F. P.: Mesenchymal stem cell therapy in joint disease. Novartis Found. Symp, 249: p. 86-96; discussion 96-102, 170-4, 239-41 (2003); Zhang H N, Li L, Leng P, Wang Y Z, Lv C Y. Uninduced adipose-derived stem cells repair the defect of full-thickness hyaline cartilage. Chin J Traumatol 2009; 12:92-97.). In 2008, Centeno and his group have reported regeneration of knee cartilage in a human by using autologous, culture-expanded bone marrow-derived stem cells (Centeno C J, Buse D, Kisiday J, Keohan C, Freeman M, Karli D: Increased Knee Cartilage Volume in Degenerative Joint Disease using Percutaneously Implanted, Autologous Mesenchymal Stem Cells. Pain Physician 11:3:343-353 (2008)).

Korean patent publication No. 10-2003-15160 discloses a composition comprising cell ingredients (mesenchymal stem cells, precursor cells derived from the mesenchymal stem cells that can be differentiated to cartilage cells or bone cells), medium and biocompatible polymer for treating articular cartilage damage.

Korean patent publication No. 10-2005-64068 discloses a method for the use of composition for treating cartilage, comprising a) a step of preparing chondrocyte ingredients, isolated and proliferated from the cartilage of a host, like human etc.; b) a step of preparing thrombin; c) a step of preparing fibrinogen matrix; d) a step of processing cartilage damage; e) a step of collecting periosteum; f) a step of stitching up the periosteum to the damage; and g) a step injecting the composition consisting of chondrocyte ingredients, thrombin, and fibrinogen matrix to the damaged region positioned in the inside of the periosteum.

Korean patent No. 10-803576 discloses a transplanting composition for plastic surgery comprising adipose stem cells, adipose cells, adipose tissue or the mixture thereof, and biodegradable stabilizers selected from the group consisting of hyaluronic acid and collagen in a physiologically suitable buffer liquid.

U.S. Pat. No. 7,803,787 discloses compositions and methods of use thereof for treating connective tissue damage in man and animals. For example, osteoarthritis, rheumatoid arthrithis, osteochondrosis dessicans, cartilage damage, joint inflammation, joint synovitis, joint injuries, degenerative joint disease, etc., wherein, the composition comprises a therapeutically effective amount of chondroitin sulfate, N-acetyl D-glucosamine, and hyaluronan.

U.S. Pat. No. 7,078,232 discloses methods and compositions for the use of adipose tissue derived adult stem or stromal cells in combination with biocompatible, restorable and non-resorbable materials for the repair of articular cartilage fractures, wherein the compositions of the biocompatible liquid is selected from the group consisting of alginate, collagen, fibrin, hyaline, or plasma.

Also, U.S. Pat. Nos. 7,033,587, 6,429,013, and 6,841,150 disclose compositions and/or methods, wherein adipose tissue-derived stromal cells are differentiated chondrocytes for the treatment cartilage repair, but not particularly related to the present invention.

Osteonecrosis is a disease where there is cellular death (necrosis) of bone components due to interruption of the blood supply. The disease interrupts rotation or bending of articular parts, and mainly occurs in growth plate or the lower parts of an articular.

Korean patent No. 10-0278905 discloses a composition for the treatment of defects in bone comprising a matrix or matrix-forming material used to fill a defect in bone, angiogenic factor at an appropriate concentration to stimulate the formation and ingrowth of blood vessels and associated cells in the matrix and the area of the defect, and an osteogenic factor associated with a delivery system and at an appropriate concentration such that upon delivery of the osteogenic factor to cells in the matrix and defect, the cells differentiate into bone cells, wherein the angiogenic factor is selected from the group consisting of bFGF, a mixture of bFGF and heparin sulfate, TGF-beta, PDGF, TNF-alpha, angiogenin, angiotropin or combinations thereof.

Korean patent publication No. 10-2007-0121669 discloses pharmaceutical compositions for the use of inducing bone and/or cartilage formation in wound healing and tissue repair which comprises a NELL peptide or a NELL RNA. The invention discloses a method of identifying a modulator of a receptor of a NELL related peptide, comprising: contacting a receptor molecule of a NELL peptide with a test compound, contacting the NELL peptide with the receptor molecule and the test compound, detecting the extent of binding of the NELL peptide to the receptor molecule with the test compound, comparing the extent of binding of the NELL peptide to the receptor molecule with the test compound with the extent of binding of a control wherein the control is obtained by detecting the extent of binding of the NELL peptide to the receptor molecule without the test compound, and designating the test compound as a modulator of the receptor of the NELL peptide if the extent of binding of the NELL peptide to the receptor molecule with the test compound is different from the extent of binding of the control.

U.S. Pat. No. 7,807,461 relates to multipotent adult stem cells derived from human adipose tissue, particularly a method for differentiation the adult stem cells into nerve cells, fat cells, cartilage cells, osteogenic cells and insulin-releasing pancreatic beta-cells, a cellular therapeutic agent for treating osteoarthritis, osteoporosis, and diabetes, and a cellular therapeutic agent for forming breast tissue.

U.S. Pat. No. 6,391,297 discloses a method of differentiating adipose stromal cells into osteoblasts, comprising: culturing said cells in a composition which comprises a medium capable of supporting the growth of fibroblasts and differentiation inducing amounts of 13-glycerophosphate and ascorbic acid and/or ascorbic-2 phosphate.

Even though there have been a lot of researches and patent applications regarding bone regeneration and articular cartilage regeneration until now, there have been no such successful results as those of the present invention.

### SUMMARY OF THE INVENTION

The present invention provides a pharmaceutical composition for treatments, preventions, or alleviation of bone disease.

The present invention is provides a method for treating, preventing, or alleviating bone disease.

The present invention provides a pharmaceutical composition for treatment, prevention, or alleviation of articular cartilage disease.

The present invention is provides a method for treating, preventing, or alleviating articular cartilage disease.

### BRIEF DESCRIPTION OF DRAWINGS

FIG. 1 shows the pictures taken by Magnetic Resonance Imaging (MRI) before the surgical procedure and 3 months after the surgical procedure.

FIG. 2 shows the pictures taken by MRI before the surgical procedure and 3 months after the surgical procedure.

FIG. 3 shows the pictures taken by MRI before the surgical procedure and 3 months after the surgical procedure.

FIG. 4 shows the pictures taken by MRI before the surgical procedure and 3 months after the surgical procedure.

### DETAILED DESCRIPTION OF THE INVENTION

While the making and using of the various embodiments of the present invention are discussed in detail below, it should be appreciated that the present invention provides many applicable inventive concepts which can be embodied in a wide variety of specific contexts. The specific embodiments described herein are merely illustrative of specific ways to make and use of the invention and do not delimit the scope of the present invention nor the scope of the claims appended hereto.

As used herein, bone refers to a calcified connective tissue primarily comprising a network of deposited calcium and phosphate in the form of hydroxyapatite, collagen (predominantly type I collagen) and bone cells, such as osteoblasts and osteoclasts.

As used herein, cartilage refers to a type of connective tissue that contains chondrocytes embedded in an intercellular material (often referred to as the “cartilage matrix”) comprising fibrils of collagen (predominantly type II collagen along with other minor types, e.g., types IX and XI), various proteoglycans (e.g., chondroitinsulfate-, keratansulfate-, and dermatansulfate proteoglycans), other proteins, and water. Cartilage as used herein includes articular and meniscal cartilage. Articular cartilage covers the surfaces of the portions of bones in joints and allows movement in joints without direct bone-to-bone contact, and thereby prevents wearing down and damage to apposing bone surfaces. Most normal healthy articular cartilage is also described as “hyaline”, i.e., having a characteristic frosted glass appearance. Meniscal cartilage is usually found in joints which are exposed to concussion as well as movement. Such locations of meniscal cartilage include the temporo-mandibular, sterno-clavicular, acromio-clavicular, wrist and knee joints [Gray's Anatomy (New York: Bounty Books, 1977)].

Adipose-Derived Stem Cells (ADSCs) refers to multipotent stromal cells or stem cells that originate from adipose tissue and are capable of self-renewal. By “adipose” is meant any fat tissue. The adipose tissue may be brown or white adipose tissue, derived from subcutaneous, omental/visceral, mammary, gonadal, or other adipose tissue site. Preferably, the adipose tissue may be from any organism having fat tissue. Such cells may comprise a primary cell culture or an immortalized cell line. The adipose tissue may be from any organism having fat tissue.

Platelet-rich plasma (PRP) is an enriched platelet-containing mixture, isolated from whole blood, which is resuspended in a small volume of plasma. While whole blood may contain about 95% red blood cells, about 5% platelets and less than 1% white blood cells, PRP may contain 95% platelets with 4% red blood cells and 1% white blood cells, but not limited thereto. PRP can be combined with activating agents such as thrombin or calcium chloride which activate the platelets to release their contents such as cytokinins and other growth factors. PRP has been used in medicine, primarily in bone grafting and dental implant applications and as part of a composition to use as a surgical adhesive. For example, Landesberg et al (U.S. Pat. No. 6,322,785) disclose an autologous platelet gel that includes PRP for bone grafts and dental implants. The PRP is activated by collagen and is applied topically to promote wound healing. Antanavich et al. (U.S. Pat. No. 5,585,007) disclose preparation of PRP and use as a tissue sealant. Cochrum (U.S. Pat. No. 5,614,214) discloses a biopolymer that optionally includes PRP and its use to temporarily block arteries and veins. Gordinier et al. (U.S. Pat. No. 5,599,558) disclose a platelet releasate product, which includes platelets buffered to approximately pH 6.5, for use in a topical application to wounds.

Well known substance which aids rejuvenation and treatment of such ailments as connective tissue disorder is a substance known as Hyaluronic Acid (also known as “Hyaluronan” or HA). It is well known that the human body naturally contains such HA, as it is found in several parts of the body such as the soft soft connective tissue, the vitreous body of the eye, hyaline cartilage, synovial joint fluid, the dermis, and the epidermis. Within these parts of the body, HA acts as a lubricant between connective tissues of the skin, protects the joints by providing shock-absorption, and helps the body retain skin moisture. Over time, however, as the body ages, the amount of HA present in the body diminishes and the body may eventually develop one of several health problems, in part due to a decrease in the presence of HA. This effect is particularly apparent for those who are over the age 50. Generally, the skin loses viscoelasticity, and wrinkles form ultimately as result of this deficiency. In the present invention, hyaluronic acid is used as a scaffold. The average molecular weight may be 200,000-400,000.

Dexamethasone has been known as a substance which promotes mesenchymal stem cells. Dexamethasone may be dexamethasone alcohol or in the form of a pharmaceutically acceptable salt or ester. Suitable salts and esters include the acetate, isonicotinoate, phenylpropionate, pivalate, t-butyl acetate, trioxaundecanoate, disodium meta-sulphobenzoate and disodium phosphate. In the present invention, dexamethasone is used as differentiation inducer of cartilage cells.

In one aspect, the present invention provides a pharmaceutical composition for bone disease comprising adipose-derived stem cells (ADSCs), platelet-rich plasma (PRP), CaCl₂, and hyaluronic acid. By using the composition, it is possible to regenerate bone without side effects, without incisions, without surgical wounds, and thus promoting fast recovery.

In another aspect, the present invention provides pharmaceutical compositions for regeneration of bone to fill the bone defects in osteonecrosis.

In still another aspect, the present invention provides a method for treating, preventing, or alleviating bone disease.

In one embodiment, the present invention provides a pharmaceutical composition for articular cartilage disease comprising adipose-derived stem cells (ADSCs), platelet-rich plasma (PRP), CaCl₂, hyaluronic acid and dexamethasone. By using the composition, it is possible to regenerate articular cartilage without side effects, without incisions, without surgical wounds, and thus promoting a fast recovery.

In another embodiment, the present invention provides a pharmaceutical composition for regenerating knee cartilage due to meniscus cartilage damage.

In still another embodiment, the present invention provides a method for treating, preventing, or alleviating articular cartilage disease.

The present invention provides a composition for bone disease treatment comprising 0.5-25 parts by weight of ADSCs, preferably 1-20 parts by weight, 0.5-25 parts by weight of PRP, preferably 0.5-15 parts by weight, 0.01-10 parts by weight of 1-5% calcium chloride, preferably 0.1-5 parts by weight, 0.01-10 parts by weight of hyaluronic acid, preferably 0.1-5.

The present invention also provides a composition for articular cartilage disease treatment further comprising 0.01-10 parts by weight of dexathasone, preferably 0.01-2 parts by weight to the above composition.

According to the inventor's research, the above ingredients and the ratio have shown the most preferable effects. Also, the most effective concentration of calcium chloride was 3%.

The amount of the compositions to be administered may be, but not limited, to be administered way, frequency, whether or not treatment or therapy of a certain disease, seriousness and history of the disease, subject's age, height, weight, health state, or other conditions. Generally, heavier subjects need to be administered the increased, higher amount.

The present invention further provides a method for the treatment of bone disease. In one embodiment, the composition comprising ADSCs, PRP, calcium chloride and hyaluronic acid is administered to a subject a pharmaceutically effective amount in need of bone treatment. In one embodiment, the composition comprising ADSCs, PRP, calcium chloride, hyaluronic acid, and dexamethasone is administered to a subject a pharmaceutically effective amount in need of articular caltilage treatment. The subject may be mammals.

At clinical trial, non-oral administration, such as injection, can be used. In case of formulation, diluents or excipients, such as filler, bulking agent, binder, wetting agent, surfactant etc. can be used. In the formulation, sterilized water, suspension agent, emulsion, freeze-drying agent, etc can be used. Non-aqueous solvent, suspension solvent vegetable oils, such as propylene glycol, polyethylene glycol, olive oil, or ethylolate can be used. Suppository material, such as witepsol, tween 61, stearic acid, lauric aldehyde, glycerol, or gelatin can be used.

Even though in the examples below only human's bone regenerations and cartilage regenerations are described, it is expected that the composition may have the same or similar effect to connective tissues in animals.

### EXAMPLES

The invention is now further described by reference to the following examples which are intended to illustrate, not to limit the scope of the invention.

This series of case-reports represents successful clinical results of regenerating bones in osteonecrosis patients and meniscus cartilage in osteoarthritis, using percutaneously implanted, autologous MSCs along with PRP, hyaluronic acid, calcium chloride (for bone regeneration) and further comprising dexamethasone (for meniscus cartilage regeneration).

### Example 1

Clinical case-reports I and II show that a combination of percutaneously injected autologous ADSCs, hyaluronic acid, PRP and calcium chloride can regenerate bones thus, filling bone defects caused by osteonecrosis in human beings.

Stem cells were obtained from adipose tissue of the abdominal origin by digesting the lipoaspirateu tissue with collagenase enzyme. These stem cells, along with hyaluronic acid, PRP and calcium chloride were injected into hip bones. Before and after of the surgical procedure were analyzed by MRI image, physical therapy, and pain score data.

Patients' MRI image showed a big difference in the size of the hip bones. Also, the results of physical therapy, pain score and functional rating index were all improved. Therefore, it is considered that surgical operation or incision could be replaced by the injection of the composition of the present invention for the regeneration of bones.

### Case I

The patient is a 29 year old Korean female with more than one year history of right hip pain. Approximately 1 year prior to the visit, the patient started having the hip pain without any history of trauma. She was seen by a physician and was diagnosed with osteoarthritis of hip, after an MRI. After taking NSAIDS for few weeks, the hip pain improved until about 1 month prior to my office visit. Again, the patient started having the hip pain radiating to the anterior region of the right knee. The pain was worse when standing up, walking and exercising. However, the pain alleviated with rest. The pain was not much relieved with NSAIDS, this time.

Repeated MRI showed osteonecrosis of femoral head, stage 4. Since there is no effective non-surgical treatment of the disease, the patient elected to receive a stem cell treatment of the present invention. At the time of initial evaluation, the patient reported moderately severe pain (VAS score 7) on rest, increased pain when standing and walking (VAS score 9).

### Liposuction Procedure:

For one week prior to the liposuction, the patient was restricted from taking corticosteroids, aspirin, NSAIDs, and oriental herb medications.

For the liposuction procedure, the patient was brought to an operating room and was placed in a supine position. Then, the patient was sedated with Propofol 2 mg IV push and 20 mg/hour rate of continuous infusion.

After cleaning the abdominal area with povodine-iodine and placing sterile drapes, an incision of approximately 0.5 cm was made about 5 cm below the umbilicus. Then, using Tumnescent solution (500 cc NS+40 cc of 2% Lidocaine+20 cc of 0.5% Marcaine+0.5 cc of Epinephrine 1:1000), the lower abdomen area was anesthetized. Next, using a 3.0 Hartman cannula, a total of 160 cc of lipoaspirates were extracted and separated by gravity. The resulting 100 cc of adipose tissue was then centrifuged at 3500 rpm for 5 minutes. The end result was approximately 40 cc of packed adipose tissue, fibrous tissue, RBC's and a small number of nucleated cells.

The digestive enzyme, collagenase, was then mixed with the centrifuged lipoaspirates at a ratio of 1:1 and digested for 30 min at 37° Celsius while rotating.

After the digestion, the lipoaspirates were centrifuged at 100 g for 3 minutes to separate the lipoaspirate and the enzyme. The left-over enzyme was then removed.

Using 500 cc D5LR, the lipoaspirates were washed three times to remove the collagenase. After each washing, the lipoaspirates were centrifuged at 100 g. After the last centrifuge, approximately 10 g of ADSCs were obtained.

### PRP Preparation:

While preparing the ADSCs, 30 cc of autologous blood was drawn with 2.5 cc of Anticoagulant Citrate Dextrose Solution (ACD) formula. This was centrifuged at 200 g for 5 min. The resultant supernatant was drawn and spun at 1000 g for 5 minutes. The supernatant was drawn and discarded. Among the resulting buffy coat, 10 g was taken and mixed with 10 g of ADSC.

To this mixture, hyaluronic acid 2 g was added as a scaffold. This PRP was again mixed with 2 g of CaCl₂ for activation of platelets at a ratio of 10:2 (PRP 10:2 CaCl2).

### Injection of the Composition:

In order to inject the mixture of stem cells and PRP, the patient was, first, placed in a lateral position with her left side down. After cleaning with povodine-iodine and draping with sterile drapes, 2% lidocaine was used to anesthetize the hip at the femoral head region. Using a 22-gauge 3½ inch needle, 17 cc mixture of ADSCs, PRP, hyaluronic acid and CaCl2 were injected into the femoral head under the ultrasound guidance.

The patient was, then, instructed to remain still with her leg elevated for 30 minutes to allow for cell attachment. As the patient was discharged to home, the patient was instructed to maintain activity as tolerated.

The patient returned for 4 additional PRP (4 cc) injections with calcium chloride (0.8 cc) every week over 1 month period.

### Results:

After the 4th week of the composition injection, the patient's pain improved more than 50%. By the 12th week, the patient's pain improved more than 70%.

### Functional Rating Index (12) and Visual Analog Score

| Outcome Measures | Pre-Injecton | 4 week post | 12 week post |
|---|---|---|---|
| Functional Rating | 15 | 12 | 8 |
| Index | | | |
| VAS | 7 | 4 | 2 |

### Physical Therapy-Range of Motion

| | | | Ab- | Ab- | | |
|---|---|---|---|---|---|---|
| | Flexion | Flexion | duction | duction | Adduction | Adduction |
| Pt Session | (deg) | VAS | (deg) | VAS | (deg) | VAS |
| Pre- | 91 | 5 | 20 | 6 | 10 | 7 |
| injection | | | | | | |
| evaluation | | | | | | |
| 4 week | 110 | 3 | 35 | 3 | 15 | 3 |
| post | | | | | | |
| injection | | | | | | |
| 12 week | 125 | 2 | 40 | 2 | 20 | 2 |
| post | | | | | | |
| injection | | | | | | |

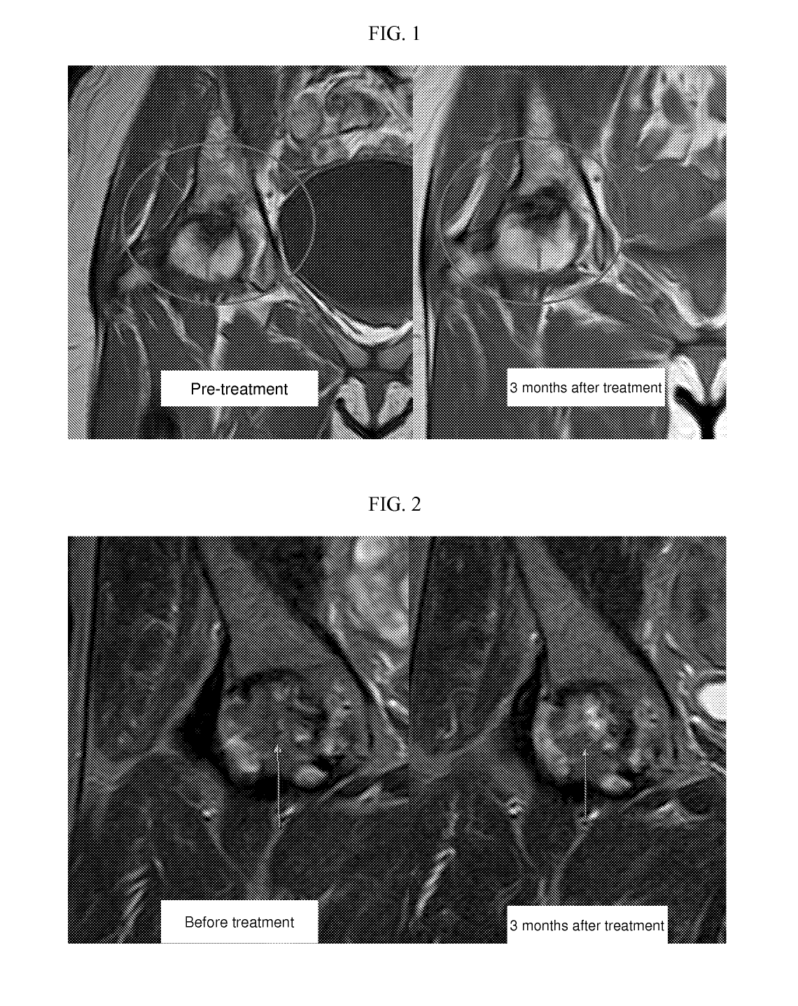

Repeated MRI taken at the 12th week showed a significant filling of bone defects on the superior acetabulum and probable bone matrix formation at the subcortical region of femoral head. (FIG. 1)

FIG. 1 is the MRI image of right hip, T1 coronal section. Lesions on superior acetabulum and subcortical head of femur has decreased in size. Bone regeneration at superior acetabulum (↓) and consolidation of bone matrix at the subcortical region of head of femur are evident ( [symbol] , ↑).

### Case II

The patient is a 47 year old Korean male who has been working as a diver until 3 years prior to the clinic visit. Approximately, 3 year ago he started having right hip pain and was diagnosed with osteonecrosis of right hip. The patient's pain has progressed over three years and the patient was offered a total hip replacement (TKR). Being reluctant with the surgical procedure, the patient elected to go with the stem cell treatment of the present invention. Before the procedure, MRI was taken and the patient was diagnosed with osteonecrosis of femoral head, stage 4.

Liposuction, PRP preparation, and the composition injection was operated as case I. The patient returned for 4 additional PRP (4 cc) injections with calcium chloride (0.8 cc) every week over 1 month period.

### Results:

After the 4th week of the present composition injection, the patient's pain improved more than 30%. However, by the 12th week, the patient's pain minimally alleviated further. Interestingly, repeat MRI taken at the 12th week showed a significant filling of bone defects with a possibility of bone matrix formation at the site of necrosis in the femoral head (FIG. 2).

### Functional Rating Index and Visual Analog Score

| Outcome Measures | Pre-Injecton | 4 week post | 12 week post |
|---|---|---|---|
| Functional Rating | 16 | 12 | 12 |
| Index | | | |
| VAS | 8 | 5 | 5 |

| | | | Ab- | Ab- | | |
|---|---|---|---|---|---|---|
| PT | Flexion | Flexion | duction | duction | Adduction | Adduction |
| Session | (deg) | VAS | (deg) | VAS | (deg) | VAS |
| Pre- | 90 | 8 | 15 | 8 | 10 | 8 |
| injection | | | | | | |
| evaluation | | | | | | |
| 4 week | 100 | 5 | 20 | 5 | 10 | 5 |
| post | | | | | | |
| injection | | | | | | |
| 12 week | 105 | 5 | 20 | 5 | 15 | 5 |
| post | | | | | | |
| injection | | | | | | |

FIG. 2 is the MRI image of right hip, T2 coronal view. Possible bone matrix regeneration is evident in the 3 months post-treatment.

### Example 2

Clinical case-reports III and IV showing a combination of percutaneously injected autologous ADSCs, hyaluronic acid, PRP, calcium chloride and dexamethasone could be able to regenerate cartilage in human osteoarthritis.

### Case III

The patient is a 70 year old Korean female with more than 5 year history of right knee pain. The patient has been a long time farm worker with active use of bilateral knee joints.

With the diagnosis of osteoarthritis of the right knee, she had received multiple injections of steroids and hyaluronic acid over the last few years. However, she did not notice any improvement of the pain. The patient was seen by an orthopedic surgeon and was offered a Total Knee Replacement (TKR). She was reluctant to go through the TKR due to possible side effects. Since then, the patient had been receiving physical therapy without much improvement.

At the time of initial evaluation, the patient reported moderately severe pain (VAS score 7) on rest, increased pain when walking. The patient also complained of mild knee swelling.

On physical examination, there was mild joint edema, decreased range of motion and tenderness with flexion. Apley's and McMurray's tests were negative, and there was no ligament laxity.

A pre-treatment 1.5 T MRI demonstrated a decreased size and deformed contour on medial meniscus of the left knee due to maceration.

Liposuction and PRP preparation was conducted as case I.

In order to inject the composition comprising 10 g of ADSCs, 10 g of PRP, 2 g of hyaluronic acid, 2 g of calcium chloride, and 1 g of dexamethasone the patient was, first, placed in a supine position with her right knee bent at 90°. After cleaning with povodine-iodine and draping with sterile drapes, the knee was anesthetized with 2% lidocaine at the medial and lateral sides of the inferior patella. Using a 22-gauge 1 inch needle, total 8.5 cc of the composition of the present invention was injected into each side of medial and lateral knee.

The patient was, then, instructed to remain still for 30 minutes to allow for cell attachment. As the patient was discharged from the clinic, the patient was instructed to maintain activity as tolerated.

The patient returned for four additional 4 cc of PRP and 0.8 cc of dexamethasone injections over 4 weeks

### Results:

After the 7th week of ADSC injection, patient's pain alleviated more than 80% and the flexion of the knee improved as well. By the 12th week, the patient's pain improved more than 90% and the range of motion also further improved.

### Functional Rating Index and Visual Analog Score

| Out Mesures | Pre-Injection | 7 week post | 12 week post |
|---|---|---|---|
| Functional Rating | 36 | 16 | 13 |
| Indes | | | |
| VAS | 7 | 2 | 1 |

### Physical Therapy-Range of Motion

| | VAS | | Flexion | Extention | Extention |
|---|---|---|---|---|---|
| PT Session | Score | Flexion(deg) | VAS | (deg) | VAS |
| Pre-injection | 7 | 110 | 7 | +3 | 1/10 |
| evaluation | | | | | |
| 7 week post | 2 | 130 | 3 | +5 | 0/10 |
| injection | | | | | |
| 12 week post | 1 | 130 | 2 | +5 | 0/10 |
| injection | | | | | |

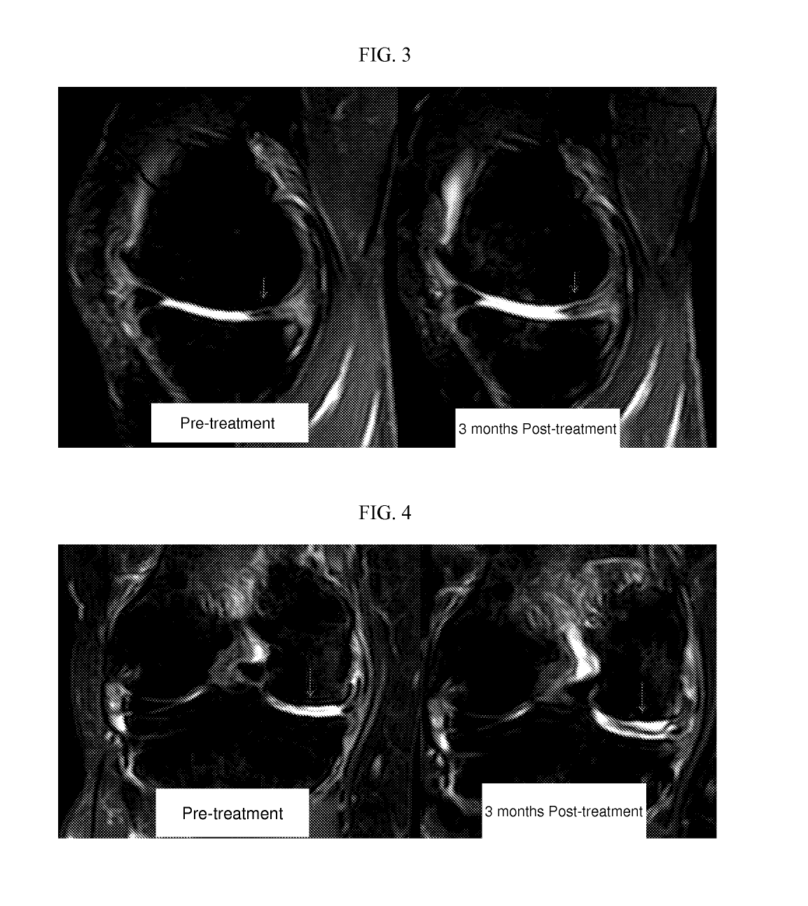

Post treatment MRI taken at the 12th week showed a significant increase in the thickness of meniscus cartilage on the medial side of the right knee (FIG. 3).

FIG. 3 is MRI Sagital T2 view. Pre- and post treatment MRI shows increased height of medial meniscus cartilage and articular cartilage (arrow).

### Case IV

The patient is a 79 year old Korean female with more than 7 year history of bilateral knee pain. The left knee is much more painful than the right. The patient has been a house wife through her life. With the diagnosis osteoarthritis of bilateral knees, she received multiple injections of steroids and hyaluronic acid on both knees over last few years. However, the patient noticed no improvement of pain. The patient was seen by an orthopedic surgeon and was offered a Total Knee Replacement (TKR). This patient was also reluctant to go through the TKR due to possible side effects. Since then, the patient was receiving physical therapy without much improvement.

At the time of initial evaluation, the patient reported severe pain on the left knee (VAS score 8) on rest, increased pain when walking.

On physical examination, there was deformity of the knee, mild joint swelling, decreased range of motion and tenderness with flexion. Apley's and McMurray's tests were negative, and there was no ligamentous laxity.

A pre-treatment 1.5 T MRI demonstrated a decreased size and deformed contour on medial meniscus of the left knee due to maceration.

Liposuction and PRP preparation and injection of the composition of case III was conducted as case III

### Results:

After the 4th weeks after ADSC injection, the patient's pain improved over 50% and the flexion of the knee improved as well. By the 12th week, the pain improved over 90% and could be able to flex the knee further.

### Functional Rating Index and Visual Analog Score

| Outcome Measures | Pre-injection | 4 week post | 12 week post |
|---|---|---|---|
| Functional Rating | 36 | 20 | 13 |
| Index | | | |
| VAS | 8 | 4 | 1 |

### Physical Therapy-Range of Motion

| | Vas | | Flexion | Extention | Extention |
|---|---|---|---|---|---|
| PT Session | Score | Flexion(deg) | VAS | (deg) | VAS |
| Pre-injection | 8 | 110 | 7 | +3 | 1/10 |
| evaluation | | | | | |
| 4 week post | 4 | 120 | 5 | +4 | 0/10 |
| injection | | | | | |
| 12 week post | 1 | 130 | 2 | +5 | 0/10 |
| injection | | | | | |

Modifications and other embodiments of the invention will become apparent to one skilled in the art to which this invention pertains having the benefit of thee teachings presented in the foregoing descriptions. It is to be understood that the invention is not limited to the specific embodiments disclosed and that modifications and other embodiments are intended to be included within the scope of the appended claims.

What is claimed is:

1. A pharmaceutical composition comprising 0.5-25 parts by weight of adipose-derived stem cells, 0.5-25 parts by weight of platelet rich plasma, 0.01-10 parts by weight of 1-5% calcium chloride, and 0.01-10 parts by weight of hyaluronic acid.

2. The pharmaceutical composition of claim 1, wherein said adipose-derived stem cells is 1-20 parts by weight, said platelet rich plasma is 0.5-15 parts by weight, said calcium chloride is 0.1-5 parts by weight, and said hyaluronic acid is 0.1-5 parts by weight.

3. A pharmaceutical composition comprising 0.5-25 parts by weight of adipose-derived stem cells, 0.5-25 parts by weight of platelet rich plasma, 0.01-10 parts by weight of 1-5% calcium chloride, and/or 0.01-10 parts by weight of hyaluronic acid, and 0.01-2 parts by weight of dexatamethasone.

4. The pharmaceutical composition of claim 3, wherein said adipose-derived stem cells is 1-20 parts by weight, said platelet rich plasma is 0.5-15 parts by weight, said calcium chloride is 0.1-5 parts by weight, and/or said hyaluronic acid is 0.1-5 parts by weight, and said dexamethasone is 0.01-2 parts by weight.

5. A method of treating, preventing, or alleviating a bone disease comprising administering to a subject a pharmaceutical effective amount of the pharmaceutical composition of claim 1.

6. The method of claim 5, wherein the bone disease is an osteonecrosis of the hip femoral head.

7. A method of treating, preventing, or alleviating a bone disease comprising administering to a subject a pharmaceutical effective amount of the pharmaceutical composition of claim 2.

8. The method of claim 7, wherein the bone disease is an osteonecrosis of the hip femoral head.

9. A method of treating, preventing, or alleviating an articular cartilage disease comprising administering to a subject a pharmaceutical effective amount of the pharmaceutical composition of claim 3.

10. The method of claim 9, wherein the articular cartilage disease is a medial cartilage of the knee.

11. A method of treating, preventing, or alleviating an articular cartilage disease comprising administering to a subject a pharmaceutical effective amount of the pharmaceutical composition of claim 4.

12. The method of claim 11, wherein the articular cartilage disease is a medial cartilage of the knee.
